(19) United States
(12) Patent Application Publication
(10) Pub. No.: US 2011/0245695 A1
(43) Pub. Date: Oct. 6, 2011

(54) ELECTRONIC SPHYGMOMANOMETER

(75) Inventors: Atsushi Kawano, Takarazuka-shi (JP); Yukiya Sawanoi, Nara-shi (JP)

(73) Assignee: OMRON HEALTHCARE CO., LTD., Kyoto-shi (JP)

(21) Appl. No.: 13/159,857

(22) Filed: Jun. 14, 2011

Related U.S. Application Data
(63) Continuation of application No. PCT/JP2009/070473, filed on Dec. 7, 2009.

(30) Foreign Application Priority Data
Dec. 17, 2008 (JP) 2008-321470

(51) Int. Cl. A61B 5/0225

(52) U.S. Cl. 600/493

(57) ABSTRACT

An electronic sphygmomanometer includes a pressure control unit configured to conduct pressurization control on a cuff (pressure control toward a specific direction) and acquire a cuff pressure signal at a time of the pressurization control to thereby measure a pressure pulse wave; a displacement decision unit configured to decide whether displacement has occurred between a position of the cuff and a virtual position of the heart based on an output from an angle sensor when the pressurization control is being conducted; and a return processing unit configured to, when it is decided that displacement has occurred, stop the pressurization control and conduct depressurization control on the cuff to thereby return an intra-cuff pressure to a specific pressure value that denotes a pressure value before the occurrence of the displacement. The pressure control unit resumes the pressurization control after processing by the return processing unit.

### TECHNICAL FIELD

The present invention relates to an electronic sphygmomanometer and, more specifically to, an electronic sphygmomanometer equipped with an angle sensor.

### BACKGROUND ART

An electronic sphygmomanometer for measuring a blood pressure based on arterial blood pressure information of an upper arm, a wrist, and a finger has been widely used. Values of the blood pressure tend to be determined lower if a measurement site is higher than the heart and determined higher if the measurement site is lower than the heart in position. Therefore, it is necessary to measure the blood pressure in a condition where the height of the measurement site agrees with that of the heart. Disagreement in height between the measurement site and the heart has been a major factor of fluctuations against management of daily changes in blood pressure value.

Accordingly, a proposal has conventionally been made to measure the blood pressure in a condition where the height of the measurement site agrees with that of the heart, as disclosed in Japanese Unexamined Patent Publication No. 2007-054648 (Patent Document 1), Japanese Unexamined Patent Publication No. 2003-102693 (Patent Document 2), and Japanese Unexamined Patent Publication No. 8-580 (Patent Document 3). For example, Japanese Unexamined Patent Publication No. 2007-054648 (Patent Document 1) discloses an electronic sphygmomanometer using a wrist as a measurement site, in which an upper arm is fitted with an angle sensor in addition to that fitted to a lower arm, to calculate a difference in height between the position of a cuff and that of the heart based on a detected angle of the lower arm and that of the upper arm. Further, it is also disclosed to detect a pitch-directional angle and a roll-directional angle of the lower arm by using a biaxial angle sensor, thereby calculating a difference in height between the cuff position and the heart position based on those detected values.

### PRIOR ART DOCUMENT PATENT DOCUMENTS

Patent Document 1: Japanese Unexamined Patent Publication No. 2007-054648
Patent Document 2: Japanese Unexamined Patent Publication No. 2003-102693
Patent Document 3: Japanese Unexamined Patent Publication No. 8-580

### SUMMARY OF INVENTION

Those conventional techniques can lead to a good measurement posture by calculating a difference in height between the position of a cuff and that of the heart. However, if the posture goes bad during measurement of the blood pressure (the difference in height between the two exceeds a predetermined range), a pressure pulse wave is disturbed, so that it is impossible to continue measurement while maintaining measurement accuracy. Therefore, in such a case, to maintain measurement accuracy, measurement has had to be repeated from the beginning.

Therefore, one or more embodiments of the present invention provides an electronic sphygmomanometer that can continue measurement while maintaining measurement accuracy if a displacement occurs between the position of a measurement site (height) and that (height) of the heart.

An electronic sphygmomanometer according to one or more embodiments of the present invention includes a cuff configured to be wound around a predetermined measurement site of a person to be measured; an angle sensor configured to detect an angle of the cuff with respect to a predetermined reference direction; a pressure sensor configured to detect a cuff pressure signal that denotes a pressure in the cuff; a pressure control unit configured to conduct first pressure control for the purpose of changing the intra-cuff pressure in a specific direction and acquire the cuff pressure signal at the time of the first pressure control to thereby measure a pressure pulse wave; a decision unit configured to decide whether displacement has occurred between a position of the cuff and a virtual position of the heart based on an output from the angle sensor when the first pressure control is being conducted; and a return processing unit configured to, if the decision unit decides that displacement has occurred, stop the first pressure control and conduct second pressure control for the purpose of changing the intra-cuff pressure in an opposite direction of the specific direction to thereby return the intra-cuff pressure to a specific pressure value that denotes a pressure value before the occurrence of the displacement, wherein the pressure control unit resumes the first pressure control after processing is performed by the return processing unit.

According to one or more embodiments of the present invention, the electronic sphygmomanometer further includes a calculation unit configured to calculate a blood pressure value based on an amplitude of the pressure pulse wave measured before occurrence of the displacement and an amplitude of the pressure pulse wave after resumption of the first pressure control.

According to one or more embodiments of the present invention, the return processing unit leads the person to be measured to correct the cuff position based on the output from the angle sensor.

According to one or more embodiments of the present invention, the electronic sphygmomanometer further includes a display unit and the return processing unit performs processing to display information denoting a positional relationship between the cuff position and the heart position on the display unit based on the output from the angle sensor.

According to one or more embodiments of the present invention, the pressure control unit resumes the first pressure control when it is detected as a result of the processing by the return processing unit that the cuff position and the heart position have fallen in a predetermined range.

According to one or more embodiments of the present invention, the pressure control unit resumes the first pressure control when it is detected as a result of the processing by the return processing unit that the cuff position and the heart position have fallen in a predetermined range and also that a shape of the wave is stabilized.

According to one or more embodiments of the present invention, the electronic sphygmomanometer further includes an informing processing unit configured to inform about a relationship between the cuff position and the heart position based on the output from the angle sensor before starting measurement and the pressure control unit starts the first pressure control when it is detected that the cuff position and the heart position have fallen in the predetermined range.

According to one or more embodiments of the present invention, the specific pressure value is a value obtained by subtracting a predetermined value from the pressure value at the time when the first pressure control is stopped. According to one or more embodiments of the present invention, the aforesaid predetermined value is larger than a difference between the pressure value at a point in time when it is decided that the displacement occurred and the pressure value at a point in time when the first pressure control is stopped.

According to one or more embodiments of the present invention, the specific pressure value is obtained by subtracting a predetermined value from the pressure value at the time when the displacement has occurred.

One or more embodiments of the present invention enables continuing measurement even if a displacement occurs between the position of a cuff and that of the heart during the measurement. Further, one or more embodiments of the present invention resumes first pressure control after returning an intra-cuff pressure to a pressure value before occurrence of the displacement in order to maintain measurement accuracy.

### BRIEF DESCRIPTION OF THE DRAWINGS

FIG. 1 is an external perspective view of a sphygmomanometer according to one or more embodiments of the present invention.

FIG. 2 is a block diagram showing a hardware configuration of the sphygmomanometer according to one or more embodiments of the present invention.

FIG. 3 is a functional block diagram showing a functional configuration of the sphygmomanometer according to one or more embodiments of the present invention.

FIG. 4 is a flowchart showing blood pressure measurement processing performed by the sphygmomanometer according to one or more embodiments of the present invention.

FIGS. 5(a) to 5(c) are illustrative views showing one example of a screen that informs about a positional relationship between a measurement site and the heart.

FIGS. 6(a) and 6(b) are illustrative views showing another example of the screen that informs about the positional relationship between the measurement site and the heart.

FIG. 7 is a flowchart showing return processing in blood pressure measurement processing according to one or more embodiments of the present invention.

FIGS. 8(a) and 8(b) are explanatory graphs of the return processing performed in the blood pressure measurement processing according to one or more embodiments of the present invention.

FIGS. 9(a) and 9(b) are tables showing one example of a data structure of pressure pulse wave-related information, which is stored in a memory unit during a period of measurement.

### DETAILED DESCRIPTION OF INVENTION

Embodiments of the present invention will be described with reference to the drawings. Identical reference numerals are given to identical and corresponding portions in the drawings, and description thereof will not be repeated.

### <External View and Configuration>

First, a description will be given of an external view and a configuration of an electronic sphygmomanometer (hereinafter referred to as “sphygmomanometer”) according to one or more embodiments of the present invention.

### (External View)

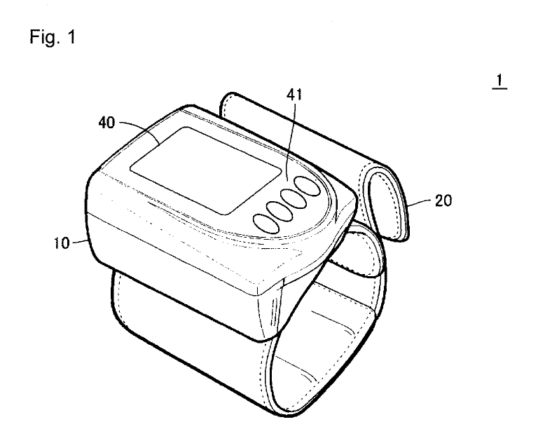

FIG. 1 is an external perspective view of a sphygmomanometer 1 according to one or more embodiments of the present invention.

As shown in FIG. 1, the sphygmomanometer 1 is equipped with a main body portion 10 and a cuff 20 that can be wound around a wrist of a person to be measured. The main body portion 10 is attached to the cuff 20. On a surface of the main body portion 10, a display unit 40 comprised of a liquid crystal display, for example, and an operation unit 41 configured to receive instructions from a user (represented by the person to be measured) are disposed. The operation unit 41 includes a plurality of switches, for example.

### (Hardware Configuration)

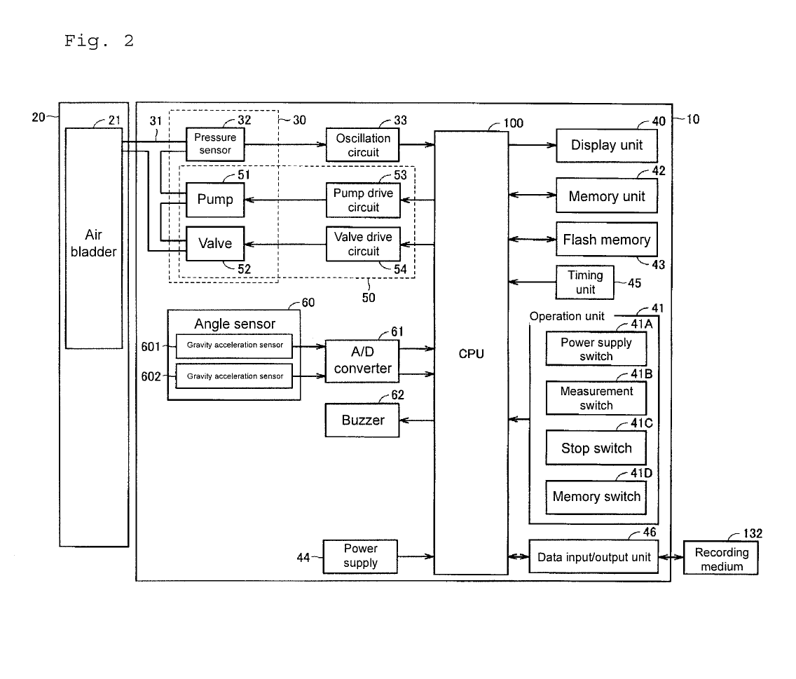

FIG. 2 is a block diagram showing the hardware configuration of the sphygmomanometer 1 according to one or more embodiments of the present invention.

As shown in FIG. 2, the cuff 20 of the sphygmomanometer 1 includes an air bladder 21. The air bladder 21 is connected to an air system 30 via an air tube 31.

The main body portion 10 includes the aforesaid display unit 40 and the operation unit 41 as well as the air system 30, a central processing unit (CPU) 100 configured to centrally control the units and perform various kinds of operation processing, a memory unit 42 configured to store a program, which causes the CPU 100 to perform predetermined operations and various kinds of data, a nonvolatile memory (flash memory, for example) 43 configured to store measured blood pressure values, a power supply 44 configured to supply power to the CPU 100, a timing unit 45 to perform timing operations, a data input/output unit 46 configured to receive incoming data from the outside, an angle sensor 60 configured to detect an angle of the main body portion 10 (attached to the cuff 20) with respect to a predetermined reference angle, an analog to digital (A/D) converter 61 configured to convert an analog signal from the angle sensor 60 into a digital signal, and a buzzer 62 configured to produce a warning beep.

The operation unit 41 has a power supply switch 41A configured to receive an incoming instruction which turns on or off the power supply, a measurement switch 41B configured to receive an instruction that starts measurement, a stop switch 41C configured to receive an instruction that stops measurement, and a memory switch 41D configured to receive an instruction that reads information such as blood pressures recorded in the flash memory 43. The operation unit 41 may further have an ID switch (not shown), which is operated to input identification (ID) information that identifies the person to be measured. This enables recording and reading measured data for each person to be measured.

The air system 30 includes a pressure sensor 32 configured to detect a pressure (cuff pressure) in the air bladder 21, a pump 51 configured to supply air to the air bladder 21 in order to pressurize the cuff, and a valve 52 that is opened and closed to discharge air from the air bladder 21 and fill it with air.

The main body portion 10 further includes an oscillation circuit 33, a pump drive circuit 53, and a valve drive circuit 54 in association with the aforesaid air system 30.

The pressure sensor 32 is of a capacitance type and changes in capacitance value with the cuff pressure. The pressure sensor 32 is not limited to a capacitance type and may be of a piezo-resistance type. The oscillation circuit 33 provides the CPU 100 with a signal having an oscillating frequency that corresponds to a capacitance value of the pressure sensor 32. The CPU 100 converts the signal supplied from the oscillation circuit 33 into a pressure, thereby detecting the pressure. The pump drive circuit 53 conducts opening/closing control on the valve 52 based on a control signal supplied from the CPU 100.

The pumps 51, the valve 52, the pump drive circuit 53, and the valve drive circuit 54 constitute an adjustment unit 50 configured to adjust the cuff pressure. A device for adjusting the cuff pressure according to one or more embodiments of the present invention is not limited to those.

The data input/output unit 46 reads the program and data from and writes them to an attachable/detachable recording medium 132, for example. And/or, the data input/output unit 46 may be able to send to and receive the program and data from an external computer (not shown) via a communication line.

The angle sensor 60 is of a biaxial type, for example, and includes a pitch-directional gravity acceleration sensor 601 and a roll-directional gravity acceleration sensor 602. In this case, the “reference direction” that provides a reference for angle detection is the vertical direction, for example.

The A/D converter 61 receives signals from the two gravity acceleration sensors 601 and 602 and converts them into digital signals. Then, the A/D converter 61 supplies the converted digital signals to the CPU 100 independently of each other. This enables the CPU 100 to calculate a displacement value (represented by a difference in height) between a position of the wrist, which is a measurement site, and a virtual position of the heart.

The angle sensor 60 is not limited to such a biaxial one and may be a uniaxial one as long as the angle sensor can detect an angle of the cuff 20 wound around the wrist. Alternatively, an angle sensor may be fitted not only to the main body portion 10 but also to the upper arm separately as disclosed in Japanese Unexamined Patent Publication No. 2007-054648 (Patent Document 1).

Further, although in the sphygmomanometer 1 according to one or more embodiments of present invention the main body portion 10 is fitted to the cuff 20 as shown in FIG. 1, the main body portion 10 and the cuff 20 may be separated from each other as employed in an upper arm type sphygmomanometer and connected to each other with an air tube (air tube 31 in FIG. 2). In this case, the angle sensor 60 only needs to be mounted on the cuff 20 not on the main body portion 10.

Although the cuff 20 is assumed to include the air bladder 21, the fluid supplied to the cuff 20 is not limited to air and may be a liquid or gel. Alternatively, what is supplied is not limited to a fluid and may be uniform fine particles such as micro-beads.

Further, although one or more embodiments of the present invention assumes that the predetermined measurement site is the wrist, this is not restrictive, and any other site such as the upper arm may be used instead.

### (Functional Configuration)

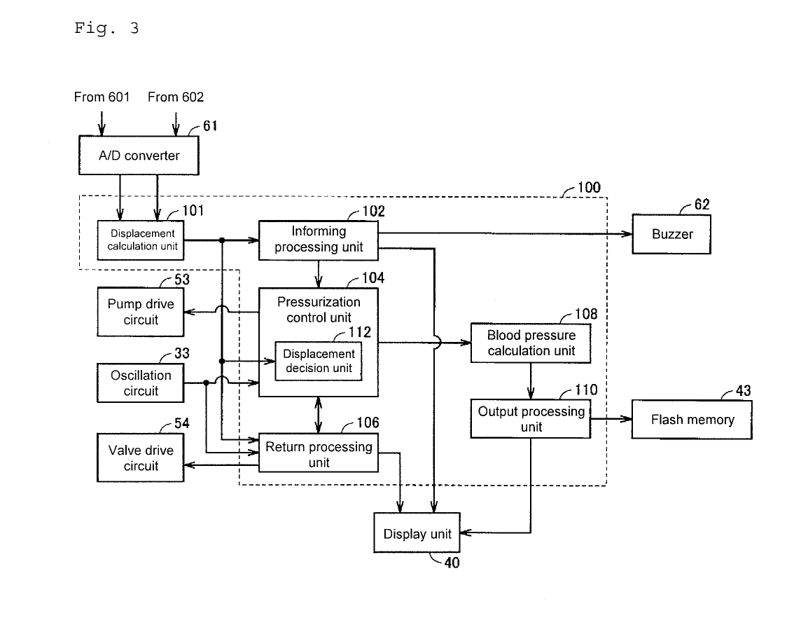

FIG. 3 is a functional block diagram showing the functional configuration of the sphygmomanometer 1 according to one or more embodiments of the present invention. FIG. 3 shows a functional configuration of a pressurization measurement type, that is, a type to calculate a blood pressure value based on a blood pressure value obtained at the time of pressurization.

As shown in FIG. 3, as its functions, the CPU 100 has a displacement calculation unit 101, an informing processing unit 102, a pressurization control unit 104, a return processing unit 106, a blood pressure calculation unit 108, and an output processing unit 110. The pressurization control unit 104 has a displacement decision unit 112. For ease of explanation, FIG. 3 only shows peripheral hardware configured to directly send signals to and receive them from the functional units of the CPU 100.

The displacement calculation unit 101 calculates a relative displacement value between a height of the wrist and a virtual height of the heart based on the detection signals of the two gravity acceleration sensors 601 and 602 input from the A/D converter 61. Specifically, first, based on the two detection signals received from the A/D converter 61, a pitch-directional angle and a roll-directional angle of the wrist are calculated. Then, based on the calculated angles, a difference in height (that is, a displacement value) between a position of the heart and a predetermined reference position of the sphygmomanometer 1 is calculated. Although one or more embodiments of the present invention have calculated the displacement value by using a method disclosed in Japanese Unexamined Patent Publication No. 2007-054648 (Patent Document 1), embodiments of the present invention are not limited to this method and can be realized by a publicly known method. The displacement calculation unit 101 outputs information of the calculated displacement value to the informing processing unit 102, the displacement decision unit 112 in the pressurization control unit 104, and the return processing unit 106.

The informing processing unit 102 performs processing to inform about a relationship between the position of the wrist and that of the heart based on the received displacement value information in order to lead the person to be measured to be in a good posture for measurement before measurement starts. Specifically, the informing processing unit 102 displays the received displacement value information on the display unit 40 or produces a warning beep by using the buzzer 62.

The pressurization control unit 104 conducts control so that the pressure in the cuff 20 may change in a specific direction, that is, an upward direction. In other words, the pressurization control unit 104 conducts pressurization control on the cuff 20 by driving the pump 51. Further, in pressurization control, the pressurization control unit 104 receives a signal from the oscillation circuit 33, to measure a pressure pulse wave. According to one or more embodiments of the present invention, the pressurization control unit 104 also calculates an amplitude of the pressure pulse wave in pressurization control.

Concurrently with the processing by the pressurization control unit 104, the decision unit 112 decides whether the height of the wrist and that of the heart have displaced by at least a predetermined value, that is, displacement has occurred between the position of the wrist and that of the heart, based on displacement value information supplied from the displacement calculation unit 101.

The return processing unit 106 stops pressurization control if it is decided by the displacement decision unit 112 that displacement has occurred and performs processing to decrease the pressure to a specific pressure value before the occurrence of the displacement. The “specific pressure value” refers to, for example, a value obtained by subtracting a predetermined value (8 mmHg, for example) from a pressure value at a time when pressurization control is stopped or a value obtained by subtracting a predetermined value (5 mmHg, for example) from a pressure value at a time when the displacement has occurred. According to one or more embodiments of the present invention, both of those two predetermined values are values that would return as much as a value that corresponds to a lapse of time of one through three pulse beats (standard lapse of time in an adult) since a point of time of the occurrence of the displacement. Those predetermined values may be set depending on a rate for pressurization control at the time of displacement, for example. If the pressurization rate can be changed with certain situations and conditions, the predetermined values are not restrictive, and a return pressure value may be calculated, which corresponds to a lapse of time of one through three pulse beats (standard lapse of time in an adult) since a point of time of the occurrence of the displacement.

If reverse-directional pressurization control is conducted by the return processing unit 106 in a partial pressure zone, pressurization control is conducted again by the pressurization control unit 104.

The blood pressure calculation unit 108 calculates blood pressure values, for example, a maximum blood pressure value and a minimum blood pressure value based on an amplitude of the pressure pulse wave calculated during pressurization control by the pressurization control unit 104. If the return processing unit 106 performs the processing in a measurement lapse of time, the blood pressure calculation unit 108 calculates the blood pressure values based on an amplitude of the pressure pulse wave measured before the occurrence of displacement and that measured after resumption of pressurization control. Information about the calculated blood pressure values is output to the output processing unit 110.

The output processing unit 110 performs processing to output the received blood pressure value information. Specifically, the output processing unit 110 displays the blood pressure values on the display unit 40, for example. Further, the output processing unit 110 may store the blood pressure values in the flash memory 43 in a condition where they are correlated with a measurement date.

Operations of those functional blocks may be realized by executing the software stored in the memory unit 42, and the operations of at least one of those functional blocks may be realized with hardware.

### <Operations>

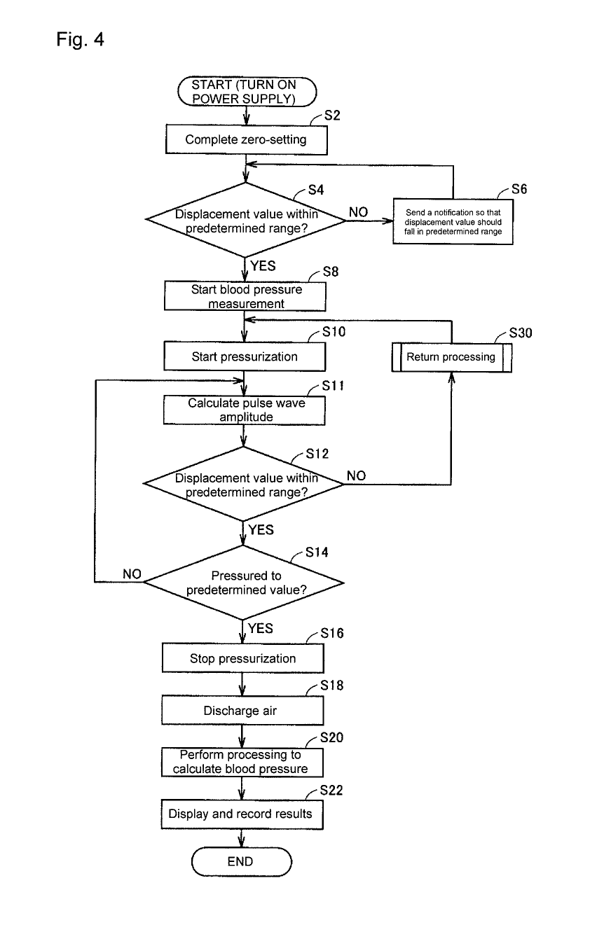

FIG. 4 is a flowchart showing blood pressure measurement processing performed by the sphygmomanometer 1 according to one or more embodiments of the present invention. The processing shown in the flowchart in FIG. 4 is stored as a program in the memory unit 42 beforehand so that the program may be read and executed by the CPU 100 to realize functions of the blood pressure measurement processing.

The processing may be started if the measurement switch 41B is pressed after the power supply is turned on. It is assumed that before the measurement switch 41B is pressed, displacement value calculation processing is performed by the displacement calculation unit 101 in another routine, and information of the calculated displacement value is overwritten and recorded in a predetermined region in the memory unit 42.

As shown in FIG. 4, first the CPU 100 completes zero setting, that is, initial reset operations (step S2). Specifically, the CPU 100 initializes a predetermined region in the memory unit 42, to discharge an air from the air bladder 21 and perform O-mmHg correction on the pressure sensor 32.

Next, the information processing unit 102 decides whether the displacement value calculated by the displacement calculation unit 101 is within a predetermined range (step S4). That is, it is decided whether a difference in height between the position of the wrist and that of the heart is at least a predetermined value. If the displacement value is not within the predetermined range (NO in step S4), the information processing unit 102 sends a notification so that the displacement value should fall in the predetermined range (step S6), to make a return to step S4. In step S6, information denoting of a difference in height, that is, a positional relationship between the height of the wrist and that of the heart or information denoting whether the height of the wrist is at a prescribed position may be displayed on the display unit 40, for example.

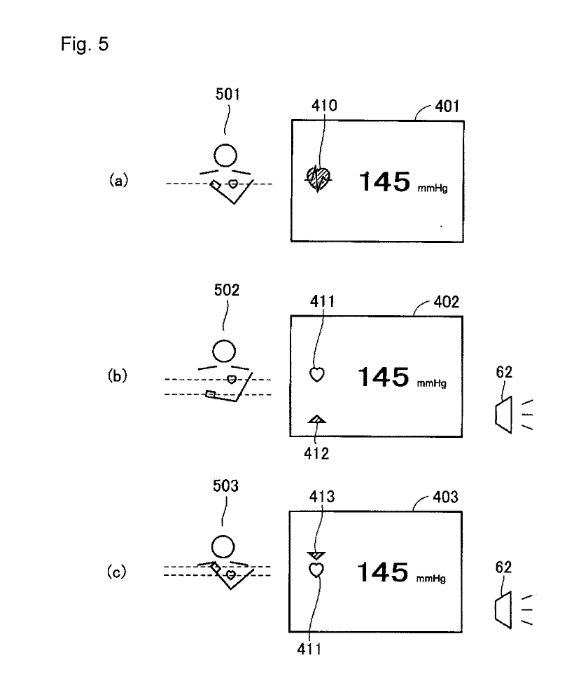

One example of an informing screen in step S6 is shown in FIGS. 5(a), 5(b), and 5(c).

As shown in FIG. 5(a), in the case of a state 501 in which the height of the wrist roughly agrees with that of the heart so that a displacement value between the two may be within the predetermined range, one mark 410 such as that shown on a screen 401 appears. The mark 410 has such a shape that one beat of the pulse wave is superimposed on a heart mark. As shown in FIG. 5(b), in the case of a state 502 in which the position of the wrist is displaced downward from that of the heart in excess of the predetermined range, an upward triangular mark 412 such as that shown on a screen 402, for example, appears below a heart mark 411. As shown in FIG. 5(c), in the case of a state 503 in which the position of the wrist is displaced upward from that of the heart in excess of the predetermined range, a downward triangular mark 413 such as that shown on a screen 403, for example, appears above the heart mark 411.

In the cases of the states 502 and 503 such as those shown in FIGS. 5(b) and 5(c), a warning beep may be produced using the buzzer 62.

In such a manner, the triangular mark 413 would match the displacement direction and be directed to have arrow functions. Furthermore, the position of the triangular mark 413 is changed with the height of the wrist with respect to the heart mark 411. Therefore, the person to be measured can intuitively know how much and in which direction he should move his wrist which the cuff 20 is wound around.

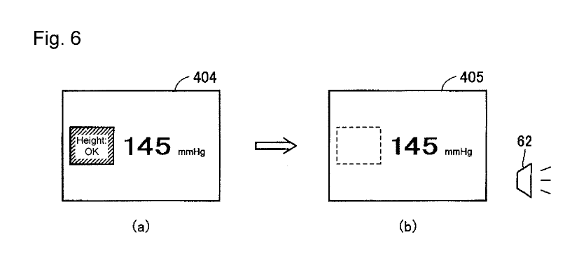

Another example of the informing screen in step S6 is shown in FIGS. 6(a) and 6(b).

As shown in FIG. 6(a), in a case where the height of the wrist roughly agrees with that of the heart (the state 501 in FIG. 5(a)), message “Height is OK” appears as shown on a screen 404. Then, if a displacement value between the two falls outside the predetermined range, message “Height is OK” disappears as shown in a screen 405 in FIG. 6(b). Along with the appearance of the screen 405 in FIG. 6(b), a warning beep may be produced using the buzzer 62.

In the case of an informing aspect such as that shown in FIG. 6, the informing processing unit 102 may change a pattern of the warning beep with a magnitude of the displacement value.

As shown in FIG. 4 again, if it is decided that the displacement value has fallen in the predetermined range (YES in step S4), blood pressure measurement is started (step S8).

First, the pressurization control unit 104 starts pressurization of the cuff 20 (step S10). Because the sphygmomanometer 1 is of the pressurization measurement type, control is conducted to gradually pressurize the cuff 20 so that an increase in pressure per unit time may be constant in order to enable calculation of blood pressures. The pressurization control unit 104 acquires a pressure pulse wave based on an output from the oscillation circuit 33 during pressurization, to calculate a pulse wave amplitude for each beat of the pressure pulse wave (step S11). Data of the calculated pulse wave amplitude is recorded in time-series in the memory unit 42 in a condition where it is correlated with the cuff pressure data. An example of the data structure of such pulse wave-related information in the memory unit 42 will be described later.

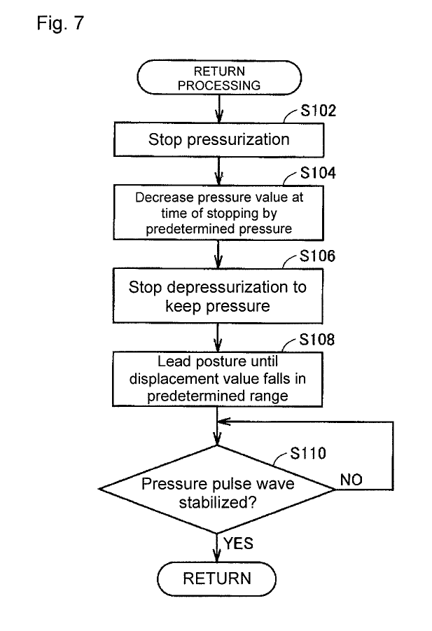

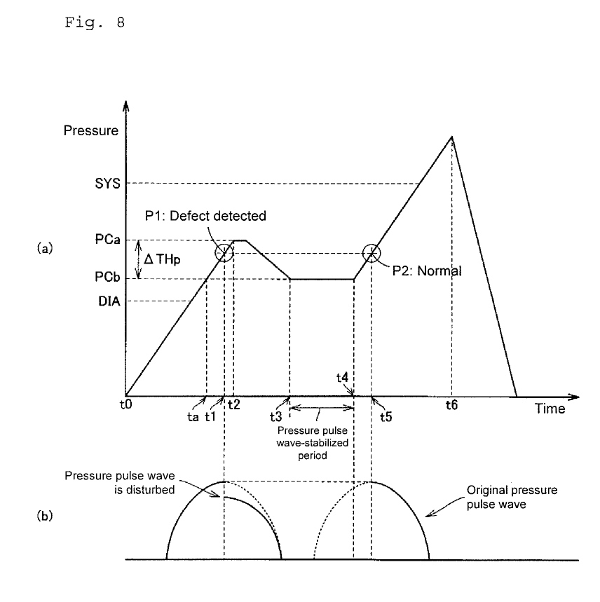

During pressurization, the displacement decision unit 112 decides whether the displacement value calculated by the displacement calculation unit 101 is within the predetermined range (step S12). If the displacement value is in the predetermined range during pressurization (YES in step S12), the shift is made to step S14. On the other hand, if the displacement value falls outside the predetermined range during pressurization, the “return processing” is performed (step S30). The return processing will be described in detail later with reference to FIGS. 7 and 8.

The pressurization control unit 104 decides in step S14 whether pressurization is performed until a predetermined value (200 mmHg, for example) is reached. If it is decided that the cuff pressure is yet to reach the predetermined value (NO in step S14), a return is made to step S11 to repeat the above processing (steps S11 and S12).

If the cuff pressure reaches the predetermined value (YES in step S14), pressurization is stopped (step S16), to discharge the air from the cuff 20 (step S18). Simultaneously, the blood pressure calculation unit 108 calculates blood pressure values (maximum and minimum blood pressures) based on the pulse wave amplitude calculated in step S11 and the corresponding cuff pressure in accordance with the oscillometric method, for example (step S20). A specific blood pressure calculation method in a case where the return processing is performed in the middle of measurement will be described later.

Finally, the output processing unit 110 displays and records results of the measurement, that is, the calculated maximum and minimum blood pressures.

Although according to one or more embodiments of the present invention, it has been assumed to continue pressurization control until the predetermined value would be reached, in the case of calculating the blood pressure values in real time, pressurization may be stopped at a point in time when the maximum blood pressure is calculated.

### (Return Processing)

Now, a description will be given of the return processing according to one or more embodiments of the present invention.

FIG. 7 is a flowchart showing the return processing in the blood pressure measurement processing according to one or more embodiments of the present invention.

As shown in FIG. 7, first the return processing unit 106 stops pressurization of the cuff 20 (step S102). That is, driving of the pump 51 is stopped.

Next, the return processing unit 106 gradually opens the closed valve 52 to decrease the pressure value at a point in time when measurement was stopped by a predetermined pressure (step S104).

After decreasing the cuff pressure by the predetermined pressure, the return processing unit 106 stops depressurization by closing the valve 52 to maintain the pressure (step S106).

If the cuff pressure is returned in such a manner to the pressure value at the point in time earlier than when the displacement occurred, the return processing unit 106 leads a posture until the displacement value calculated by the displacement calculation unit 101 falls in the predetermined range (step S108) and decides whether the pressure pulse wave is stabilized (step S110). The posture may be led by, for example, performing similar processing as the informing processing in step S6 described above. That is, the processing is performed to display the information denoting the positional relationship between the height of the wrist and that of the heart. This permits the person to be measured to correct the position of the wrist to which the cuff 20 is wound around in such a manner that his measurement posture may be good. That is, a difference in height between the wrist and the heart may fall in a predetermined value range.

Then, if the pressure pulse wave is stabilized (YES in step S110), a return is made to the main routine. Whether the pressure pulse wave is stabilized can be detected by deciding whether, for example, the amplitude value of the pressure pulse wave is roughly stabilized. That is, a difference between the previous and the following amplitude values is at most a predetermined value a predetermined number of times in a row. In such a manner, by restarting pressurization after detecting that the pressure pulse wave is stabilized, the accuracy of the blood pressure values can be improved.

Conventionally, measurement would be terminated with an error display because measurement accuracy could not be maintained if the posture went bad during measurement. However, according to one or more embodiments of the present invention, even if the posture goes bad during measurement, the person to be measured can be led to correct the position of the wrist, to avoid the measurement from being stopped. Accordingly, the person to be measured need not perform measurement all over again from the beginning, so that a total time for measurement can be reduced.

The above-described return processing will be described more specifically with reference to FIGS. 8(a) and 8(b).

FIGS. 8(a) and 8(b) are explanatory graphs of the return processing performed in the blood pressure measurement processing according to one or more embodiments of the present invention. FIG. 8(a) shows a pressure value obtained based on a signal from the oscillation circuit 33 along an axis of a time measured by a timing unit 45. FIG. 8(b) shows images of the pressure pulse waves at two feature points P1 and P2, respectively, in FIG. 8(a).

As shown in FIG. 8(a), it is assumed that pressurization control is started (whose start time is denoted by time t0), and a defect (displacement) is detected at time t1 (step S12). A pressure pulse wave measured at time t1 has a disturbed waveform as shown in FIG. 8(b), in contrast to a hitherto-existing normal one.

If displacement occurs, pressurization control on the cuff 20 is stopped (step S102). There may be some time lag from a point in time when a defect is detected until a point in time when pressurization is stopped completely, so that it is assumed that pressurization is stopped at time t2. A pressure value at this point in time is given by “PCa”.

Assuming that the predetermined pressure value by which the pressure value PCa is decreased is given by “ΔTHp”, the pressure value is gradually decreased to the pressure value “PCb” obtained by subtracting the predetermined pressure ΔTHp from the pressure value PCa at the time of stopping (step S104). A point in time when the pressure value is decreased to the pressure value PCb is assumed to be given by t3. Taking into account a time lag, the predetermined pressure ΔTHp may be a value lowered by a constant value than a pressure value at the feature point P1 where the displacement occurred, so that the pressure value “PCb” may be lower than the pressure value at the feature point P1 by the constant value.

Once the pressure is decreased to the pressure value PCb, this pressure value PCb is maintained until the pressure pulse wave is stabilized (step S106). If stability of the pressure pulse wave is detected in a time lapse between time t3 and t4 (YES in step S110), pressurization is started again (step S10). At the feature point P2 (time t5) having the same pressure level as the feature point P1 where the displacement occurred, the measured pressure pulse wave takes on its original normal waveform as shown in FIG. 8(b).

Assuming that a point in time when the pressure value was PCb under pressurization control before detection of the defect is given by time ta, if the return processing such as that shown in FIG. 8(a) is inserted in the middle of measurement, the blood pressure calculation unit 108 can calculate a maximum blood pressure (“SYS” in the figure) and a minimum blood pressure value (“DIA” in the figure) based on an amplitude value based on the pressure pulse wave information detected from time t0 to time ta and an amplitude value based on the pressure pulse wave information detected from time t4 to time t6.

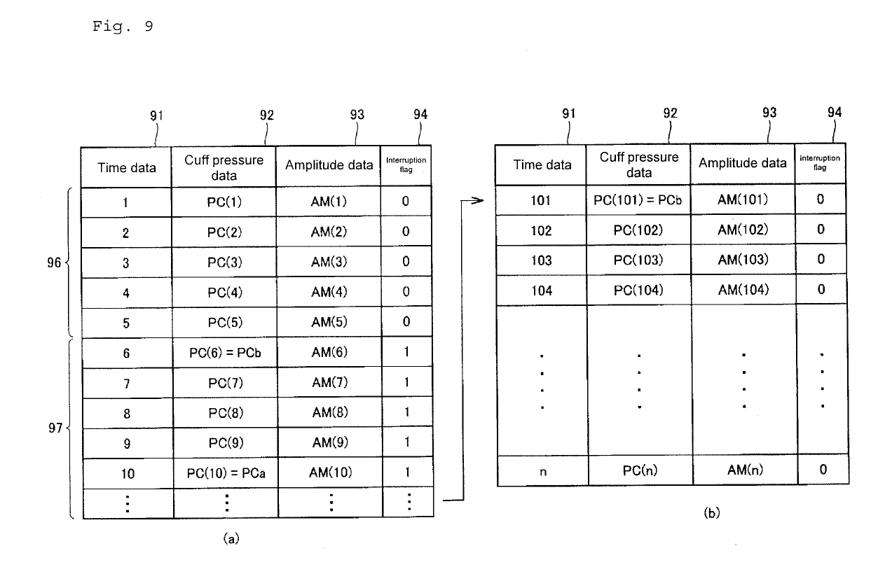

Now, a description will be given of a blood pressure calculation method by the blood pressure calculation unit 108 in a case where the return processing is performed in the middle of measurement, with reference to FIGS. 9(a) and 9(b).

FIGS. 9(a) and 9(b) are tables showing one example of a data structure of the pressure pulse wave-related information, which is stored in the memory unit 42 during a period of measurement.

As shown in FIGS. 9(a) and 9(b), the pressure pulse wave-related information contains four items; that is, an item 91 denoting time data, an item 92 denoting cuff pressure data, an item 93 denoting calculated pulse wave amplitude data, and an item 94 of an interruption flag. The cuff pressure data denotes a cuff pressure as a control value, and the pulse wave amplitude data denotes a pulse wave amplitude value calculated in step S11. The time data and the cuff pressure data and the pulse wave amplitude data are stored in a condition where they are correlated with each other. The pulse wave amplitude data only needs to be correlated with the cuff pressure data. It is assumed that numerals including “0” also are recorded as the pulse wave amplitude data.

The interruption flag (1 or 0) is used to assess whether the corresponding pulse wave amplitude value is to be used in the blood pressure calculation. The interruption flag only needs to be stored in a condition where it is correlated with the time data (or the cuff pressure data), for example, and has a stored initial value of “0”.

FIG. 9(a) shows the pressure pulse wave-related information from a point in time when measurement (pressurization) is started to a point in time when re-pressurization is started (t0 to t4), and FIG. 9(b) shows the pressure pulse wave-related information from a point in time when re-pressurization is started to a point in time when measurement is ended (t4 to t6). In FIG. 9(b), the time data at a point in time when re-pressurization is started is assumed to be “101” as an example.

As shown in FIG. 9(a), it is assumed that pressurization is stopped at a time of time data “10”. In this case, the cuff pressure data PC(10) corresponding to the time data “10” gives the pressure value PCa in FIG. 8(a).

It is supposed that a pressure value obtained by subtracting the predetermined pressure ΔTHp from the cuff pressure data PC(10) is a value of the cuff pressure data PC(6), for example. Then, in a lapse of time from PC(6) onward until re-pressurization is performed, that is, from time data “6” to time data “100”, the interruption flag is held to “1”.

In such a case, among the FIG. 9(a) data pieces, a data group 97 of the time data “6” and the subsequent time data where the interruption flag is “1” is not used in blood pressure calculation. The blood pressure calculation unit 108 calculates blood pressure values by using such time data that the interruption flag is “0”. That is, the blood pressure value is calculated by splicing a data group 96 of time data pieces “1” through “5” in FIG. 9(a) and all the data in FIG. 9(b). More specifically, a maximum blood pressure value and a minimum blood pressure value are calculated based on the values of amplitude data pieces AM(1) through AM(5) in FIG. 9(a) and amplitude data AM(101) and the subsequent amplitude data in FIG. 9(b).

In such a manner, according to one or more embodiments of the present invention, even if the posture goes bad in the middle of measurement, only the amplitude data for the good posture is used in the blood pressure calculation, making it possible to maintain good measurement accuracy.

Although one or more embodiments of the present invention have calculated a blood pressure value by using a pulse wave amplitude calculated under pressurization control, the blood pressure value may be calculated by recording only cuff pressure data (pressure pulse wave data) under pressurization control and, after the end of measurement, calculating the pulse wave amplitude from the spliced cuff pressure data.

Although one or more embodiments of the present invention have decreased the pressure value at a stopping place by the predetermined value (8 mmHg, for example), a pressure value at a time when displacement occurred may be decreased by a predetermined value (5 mmHg, for example).

Alternatively, in contrast to embodiments of the present invention wherein the pressure value at the stopping place has been decreased by the predetermined value, a pressure value at a time, for example, three pulse beats earlier may be estimated so that the pressure value would be decreased to this estimated value. The pressure value at the time three pulse beats earlier can be estimated from a lapse of time that elapses for each pulse beat of the person to be measured. In this case, a pressure value at a time that is a predetermined number of pulse beats earlier than that when the displacement occurred corresponds to the aforesaid specific pressure value.

Alternatively, an amplitude of a pressure pulse wave may be calculated at the time of depressurization so that depressurization would be stopped at a time when a difference between a pressure pulse wave amplitude value immediately before occurrence of displacement and this calculated amplitude value decreases to a predetermined value or less. In this case, a pressure value whose difference from the amplitude value immediately before the occurrence of the displacement is the predetermined value or less corresponds to the aforesaid specific pressure value.

Although embodiments of the present invention have been described with reference to the increasing pressure measurement method, one or more embodiments of the present invention can be applied also to the decreasing pressure measurement method. That is, if depressurization is stopped due to occurrence of a defect, a pressure value at the time of the stopping may be increased by a predetermined pressure until the height of the wrist is corrected to then resume depressurization.

Further, according to one or more embodiments of the present invention, the return processing has been performed irrespective of a pressure value at the time of occurrence of displacement, so that if pressurization is stopped at a high pressure value, the person to be measured may possibly feel a pain. Accordingly, if the displacement occurs at a pressure value higher than a predetermined value (130 mmHg, for example), an error display may be provided to prompt the person to be measured to measure the blood pressure again.

Alternatively, if the displacement occurs in a pressure zone (from 60 mmHg to 150 mmHg) presumed to be used in blood pressure calculation, an error display may be provided to prompt the person to be measured to measure the blood pressure again so that measurement accuracy may be improved further.

Alternatively, an air may be discharged to stop measurement if a lapse of time from stopping of pressurization (depressurization) upon detection of a defect up to re-pressurization (re-depressurization) exceeds a specific lapse of time. Further, this specific lapse of time may be varied with the pressure value at the time of defect detection. Specifically, this can be realized by, for example, storing a data table that correlates the pressure at the time of defect detection and the specific lapse of time with each other in the memory unit 42 beforehand.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### DESCRIPTION OF REFERENCE NUMERALS

1 electronic sphygmomanometer
10 main body portion
20 cuff
21 air bladder
30 air system
31 air tube
32 pressure sensor
33 oscillation circuit
40 display unit
41 operation unit
41A power supply switch
41B measurement switch
41C stop switch
41D memory switch
42 memory unit
43 flash memory
44 power supply
45 timing unit
46 data input/output unit
50 adjustment switch
51 pump
52 valve
53 pump drive circuit
54 valve drive circuit
60 angle sensor
61 A/D converter
62 buzzer
100 CPU
101 displacement calculation unit
102 informing processing unit
104 pressurization control unit
106 return processing unit
108 blood pressure calculation unit
110 output processing unit
112 displacement decision unit
132 recording medium
601, 602 gravity acceleration sensor

1. An electronic sphygmomanometer comprising:
a cuff configured to be wound around a predetermined measurement site of a person to be measured;
an angle sensor that detects an angle of the cuff with respect to a predetermined reference direction;
a pressure sensor that detects a cuff pressure signal that denotes a pressure in the cuff;
a pressure control unit that conducts a first pressure control that changes the intra-cuff pressure in a specific direction,
wherein the pressure control unit acquires the cuff pressure signal at a time of the first pressure control to thereby measure a pressure pulse wave;
a decision unit that decides whether a displacement has occurred between a position of the cuff and a virtual position of a heart based on an output from the angle sensor when the first pressure control is being conducted; and
a return processing unit that, if the decision unit decides that the displacement has occurred, stops the first pressure control and conducts a second pressure control that changes the intra-cuff pressure in an opposite direction of the specific direction to thereby return the intra-cuff pressure to a specific pressure value that denotes a pressure value before the occurrence of the displacement,
wherein the pressure control unit resumes the first pressure control after processing by the return processing unit.

2. The electronic sphygmomanometer of claim 1, further comprising a calculation unit that calculates a blood pressure value based on an amplitude of the pressure pulse wave measured before the occurrence of the displacement and an amplitude of the pressure pulse wave after the resumption of the first pressure control.

3. The electronic sphygmomanometer of claim 1, wherein the return processing unit leads the person to be measured to correct the cuff position based on the output from the angle sensor.

4. The electronic sphygmomanometer of claim 3, further comprising a display unit,
wherein the return processing unit performs processing to display information denoting a positional relationship between the cuff position and the heart position on the display unit based on the output from the angle sensor.

5. The electronic sphygmomanometer of claim 1, wherein the pressure control unit resumes the first pressure control when it is detected as a result of the processing by the return processing unit that the cuff position and the heart position have fallen in a predetermined range.

6. The electronic sphygmomanometer of claim 5, further comprising an informing processing unit that informs about a relationship between the cuff position and the heart position based on the output from the angle sensor before starting measurement,
wherein the pressure control unit starts the first pressure control when it is detected that the cuff position and the heart position have fallen in the predetermined range.

7. The electronic sphygmomanometer of claim 1, wherein the pressure control unit resumes the first pressure control when it is detected as a result of the processing by the return processing unit that the cuff position and the heart position have fallen in a predetermined range and also that a shape of the pressure pulse wave is stabilized.

8. The electronic sphygmomanometer of claim 7, further comprising an informing processing unit that informs about a relationship between the cuff position and the heart position based on the output from the angle sensor before starting measurement,
wherein the pressure control unit starts the first pressure control when it is detected that the cuff position and the heart position have fallen in the predetermined range.

9. The electronic sphygmomanometer of claim 1, wherein the specific pressure value is a value obtained by subtracting a predetermined value from the pressure value at a time when the first pressure control is stopped.

10. The electronic sphygmomanometer of claim 1, wherein the specific pressure value is a value obtained by subtracting a predetermined value from the pressure value at a time when the displacement occurred.

11. The electronic sphygmomanometer of claim 9, wherein the specific pressure value is larger than a difference between the pressure value at a point in time when it is decided that the displacement occurred and the pressure value at a point in time when the first pressure control is stopped.
